(19) United States
(12) Patent Application Publication
(10) Pub. No.: US 2022/0395261 A1
(43) Pub. Date: Dec. 15, 2022

(54) BONE BIOPSY DEVICE AND RELATED METHODS

(71) Applicant: Merit Medical Systems, Inc., South Jordan, UT (US)

(72) Inventors: Clark Ragsdale, Herriman, UT (US); Michael D. Ehninger, South Jordan, UT (US); Ricardo Rufino, Sandy, UT (US)

(21) Appl. No.: 17/805,746

(22) Filed: Jun. 7, 2022

Related U.S. Application Data
(60) Provisional application No. 63/209,333, filed on Jun. 10, 2021.

(51) Int. Cl. A61B 10/02; A61B 17/16; A61B 17/34

(52) U.S. Cl. CPC A61B 10/025; A61B 10/0266; A61B 17/1635; A61B 17/1628; A61B 17/34; A61B 2010/0208

(57) ABSTRACT

Devices and methods used to obtain core tissue samples are disclosed. The devices may be configured to drill into cortical bone and saw a hole into a bone lesion and/or bone marrow while obtaining the core tissue sample. The devices can include a motor and a clutch configured to rotate a trocar having a tip configured for drilling and an outer coax cannula having a trephine tip configured for sawing. The core tissue sample may be received within an inner cannula as an intermediate cannula cuts a hole in the bone lesion and/or bone marrow. The devices can include a spacer.

### RELATED APPLICATIONS

This application claims priority to U.S. Provisional Application No. 63/209,333 filed on Jun. 10, 2021 and titled, “BONE BIOPSY DEVICE AND RELATED METHODS,” which is hereby incorporated by reference in its entirety.

### TECHNICAL FIELD

The present disclosure relates generally to devices used to perform a biopsy procedure, specifically a bone biopsy procedure. More specifically, the present disclosure relates to devices used to drill into a bone to obtain a core tissue sample of a bone lesion and/or bone marrow.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of an embodiment of a bone biopsy device.

FIG. 2 is a perspective exploded view of the bone biopsy device of FIG. 1.

FIG. 3 is a perspective exploded view of a powertrain assembly and a tissue sampling assembly of the bone biopsy device of FIG. 1.

FIG. 4 is a front perspective view of the bone biopsy device of FIG. 1 in a ready state with a portion of a handle housing removed.

FIG. 5 is a rear perspective view of the bone biopsy device of FIG. 1 in a ready state with a portion of the handle housing removed.

FIG. 6A is a side view of a coax assembly of the bone biopsy device of FIG. 1.

FIG. 6B is a side view of an embodiment of an intermediate cannula of the bone biopsy device of FIG. 1.

FIG. 6C is a side view of an embodiment of an inner cannula of the bone biopsy device of FIG. 1.

FIG. 6D is a side view of an embodiment of a trocar of the bone biopsy device of FIG. 1.

FIG. 7 is a perspective exploded view of a clutch system of the bone biopsy device of FIG. 1.

FIG. 8A is a side view of the bone biopsy device of FIG. 1 ready for use.

FIG. 8B is a side view of the bone biopsy device of FIG. 1 inserted into a patient's skin and drilled through a cortical bone layer into a bone lesion and/or a bone marrow.

FIG. 8C is a side view of the bone biopsy device of FIG. 1 removed from an inserted outer coax cannula, a spacer removed, and the trocar retracted.

FIG. 8D is a side view of the bone biopsy device of FIG. 1 with the inner cannula and the intermediate cannula drilled into a bone lesion and/or bone marrow to obtain a core tissue sample.

FIG. 8E is a side view of the bone biopsy device of FIG. 1 with the inner cannula, intermediate cannula, and trocar removed from the outer coax cannula and the inner cannula extended.

FIG. 8F is a side view of the bone biopsy device of FIG. 1 with the trocar extended to eject the core tissue sample from the inner cannula.

FIG. 8G is a side view of the bone biopsy device of FIG. 1 with an aspiration device coupled to a connector of the coax assembly of FIG. 6A.

FIG. 8H is a side view of the bone biopsy device of FIG. 1 with a door opened for removal of a reusable housing.

FIG. 9A is a front perspective view of a manual trocar assembly.

FIG. 9B is a rear perspective view of the manual trocar assembly of FIG. 9A.

FIG. 10 is an embodiment of another bone biopsy device.

FIG. 11 is a front perspective view of the bone biopsy device of FIG. 10 in a ready state with a portion of a handle housing removed.

FIG. 12 is a rear perspective view of the bone biopsy device of FIG. 10 in a ready state with a portion of the handle housing removed.

### DETAILED DESCRIPTION

A bone biopsy device may include a handle, a tissue sampling assembly, a coax assembly, and a powertrain assembly. The handle may include a handle configured to hold the tissue sampling assembly, the coax assembly, and the powertrain assembly. The tissue sampling assembly can include an inner cannula coaxially and slidably disposed within an intermediate cannula. The inner cannula may extend distally from the handle and may be configured to receive a core tissue sample. The intermediate cannula can extend from the handle and its tip (e.g., trephine tip) can be configured to drill into a tissue (e.g., a lesion or bone marrow) when rotated by the powertrain assembly. A trocar with a penetrating tip may be coaxially and slidably disposed within a lumen of the inner cannula. The tissue sampling assembly may include a trocar displacement member configured to displace the trocar relative to the inner cannula from a first extended position where the trocar can drill into a bone to a retracted position to a second extended position where the trocar can eject the core tissue sample from the inner cannula. The coax assembly may be selectively detachable from the handle housing. The coax assembly may include an outer coax cannula extending distally from a coax connector. The inner and intermediate cannulae may be coaxially disposed within a lumen of the outer coax cannula. A tip of the outer coax cannula may be a cutting tip (e.g., a trephine tip) and may be configured to saw into a bone lesion and/or bone marrow. In certain embodiments, a spacer can be selectively disposed between the handle housing and the coax assembly.

The powertrain can include a power source, a motor, and a drivetrain disposed within the handle housing. The power source and motor may be selectively removable from the handle housing such that the power source and motor may be reusable components. The powertrain assembly may be configured to rotate one or more of the trocar, inner cannula, intermediate cannula, and coax assembly. In certain instances, the powertrain may include a clutch to selectively allow power rotation of the trocar, inner cannula, intermediate cannula, and coax assembly and not allow manual rotation via the handle housing. In other instances, the powertrain may include a gear box.

The bone biopsy device may be used by a practitioner to obtain a core tissue sample of a bone lesion and/or bone marrow. In other instances, the bone biopsy device may be used to obtain a core tissue sample of other tissues within a patient, such as a soft tissue sample. In use, the trocar, inner cannula, intermediate cannula, and outer coax cannula may be rotated by the powertrain assembly and drilled through a cortical bone layer adjacent into a lesion and/or bone marrow. The bone biopsy device may be removed from the outer coax cannula and the spacer removed from the handle housing and the coax assembly. The trocar may be retracted and the intermediate and inner cannulae inserted into the outer coax cannula. The intermediate and inner cannulae can be rotated by the powertrain to saw or otherwise obtain a core tissue sample of the lesion and/or bone marrow that is collected in the inner cannula. The intermediate and inner cannulae with the core tissue sample may be removed from the coax assembly. The inner cannula can be advanced to extend from the intermediate cannula and the trocar can be advanced to extend from the inner cannula to eject the core tissue sample from the inner cannula. A slot through a wall of the inner cannula may allow radial expansion of the inner cannula to facilitate core tissue sample ejection. The radial expansion allowed by the slot can also facilitate obtaining and retaining a core tissue sample as the inner cannula can flex outward and then apply an inwardly directed pressure on a core tissue sample retained therein. In certain instances, a medical device (e.g., syringe) can be coupled to a connector of the coax assembly to collect or aspirate bone marrow, blood, and/or tissue cells or to infuse or inject a substance (such as a medicament) into the patient.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another. Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

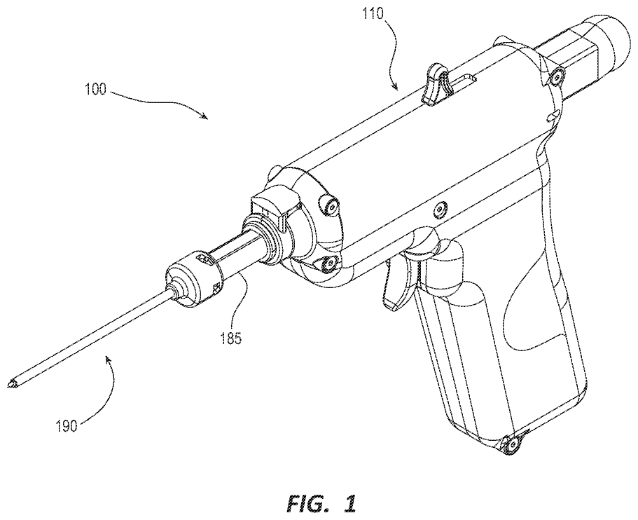

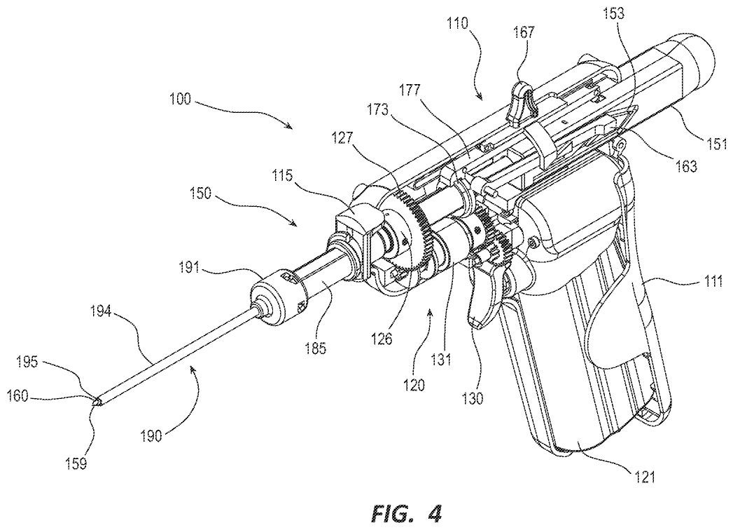

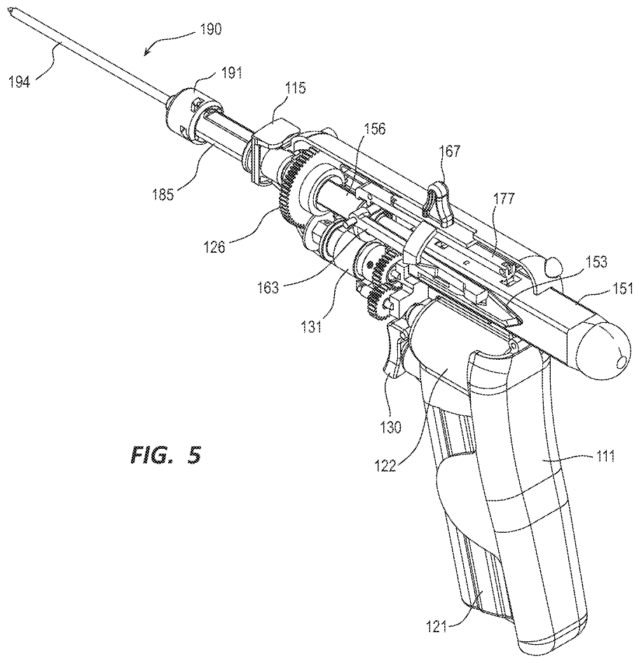

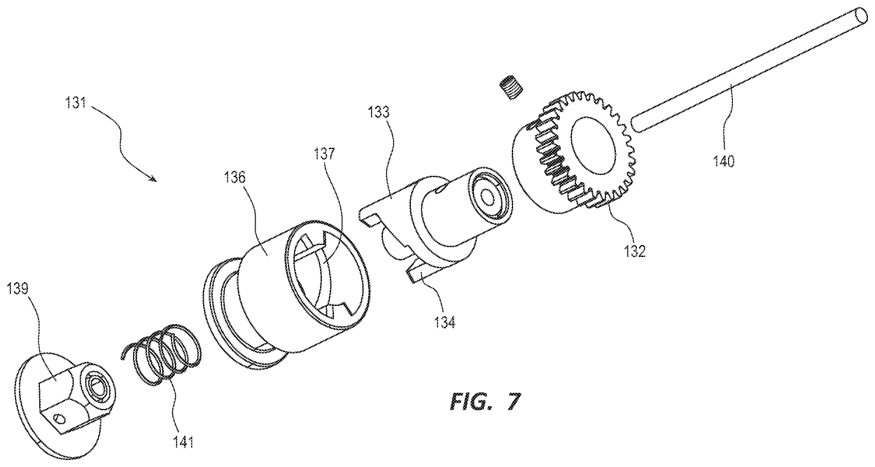

FIGS. 1-12 illustrate different views of bone biopsy devices, related components, and methods of use. In certain views each device may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

FIGS. 1-8H depict one embodiment of a powered bone biopsy device 100. FIG. 1 illustrates the bone biopsy device 100 including a handle assembly 110, a coax assembly 190, and a spacer 185 disposed between the handle assembly 110 and the coax assembly 190.

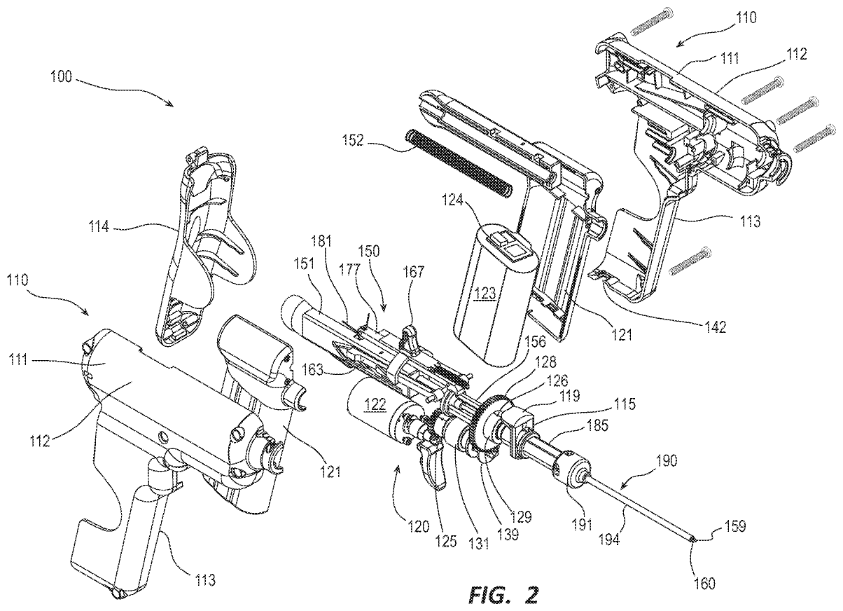

As depicted in an exploded view of the bone biopsy device 100 of FIGS. 2-5, the handle assembly 110 includes a handle housing 111, a powertrain assembly 120, and a tissue sampling assembly 150. The handle housing 111 can include an upper portion 112 and a grip portion 113. The grip portion 113 may be configured to be grasped by a hand of a practitioner during use of the bone biopsy device 100.

The handle housing 111 may be formed of two separate halves that may be coupled using any suitable technique. For example, in the illustrated embodiment of FIG. 2, the separate halves are coupled using a plurality of fasteners. In other embodiments, the separate halves may be coupled using a snap fit, welding, gluing, bonding, etc. The handle housing 111 may include any suitable polymeric and/or metallic material, such as polycarbonate, acrylonitrile butadiene styrene, polycarbonate acrylonitrile butadiene styrene copolymer, nylon, acetal, polyethylene (e.g., such as high-density polyethylene and/or low-density polyethylene), silicone, thermoplastic elastomers, steel, stainless steel, aluminum, ceramic, and combinations thereof. The polymers may also be reinforced with other materials, such as glass or aramid fibers. The handle housing 111 may be formed using any suitable technique, such as injection molding, thermoforming, machining, 3D printing, etc. The handle housing 111 can include a plurality of pockets or recesses configured to hold or retain at least some of the components of the handle assembly 110.

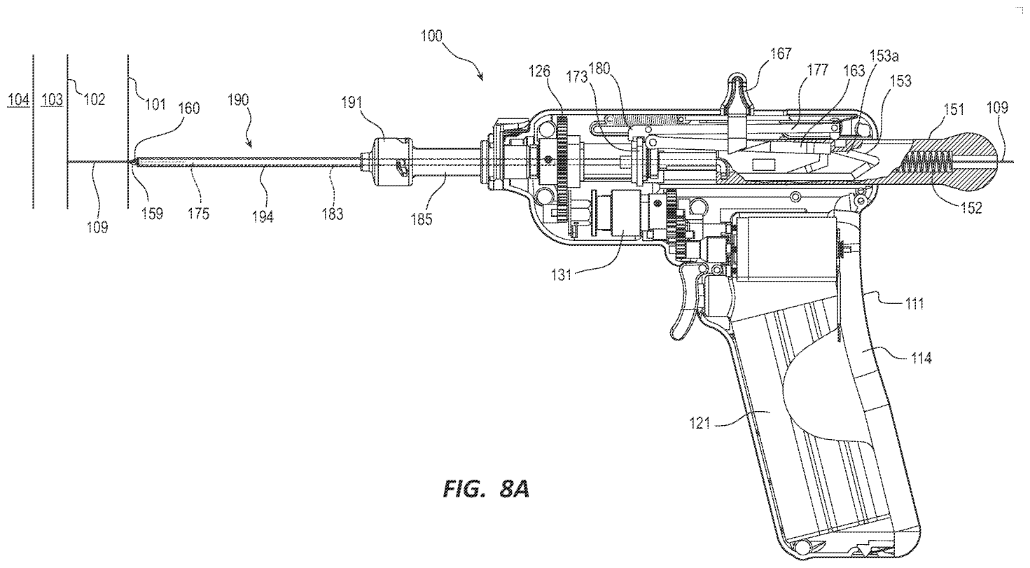

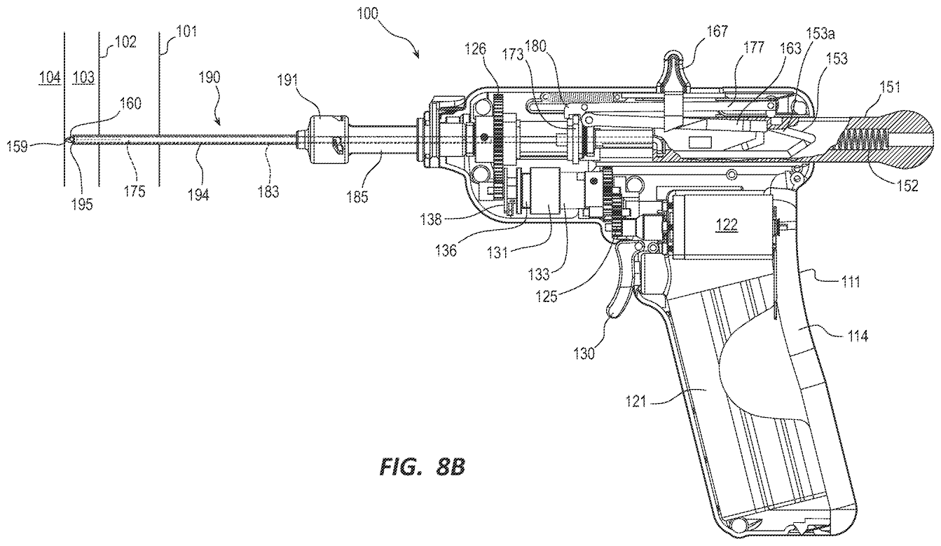

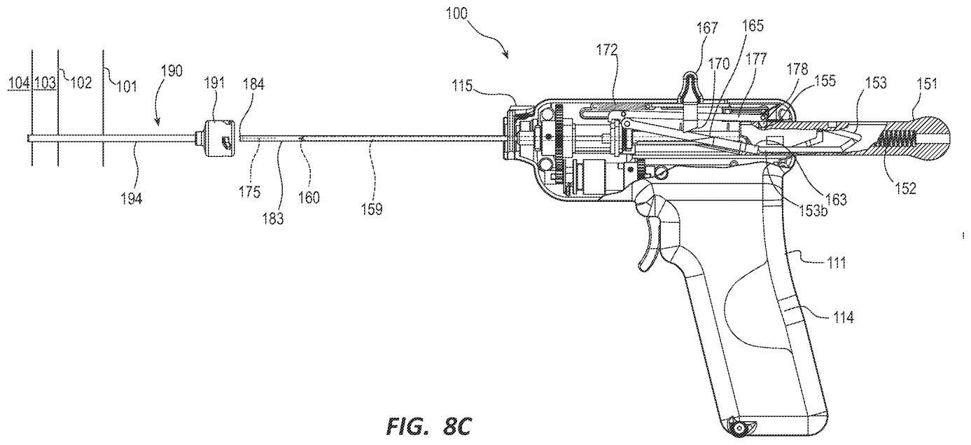

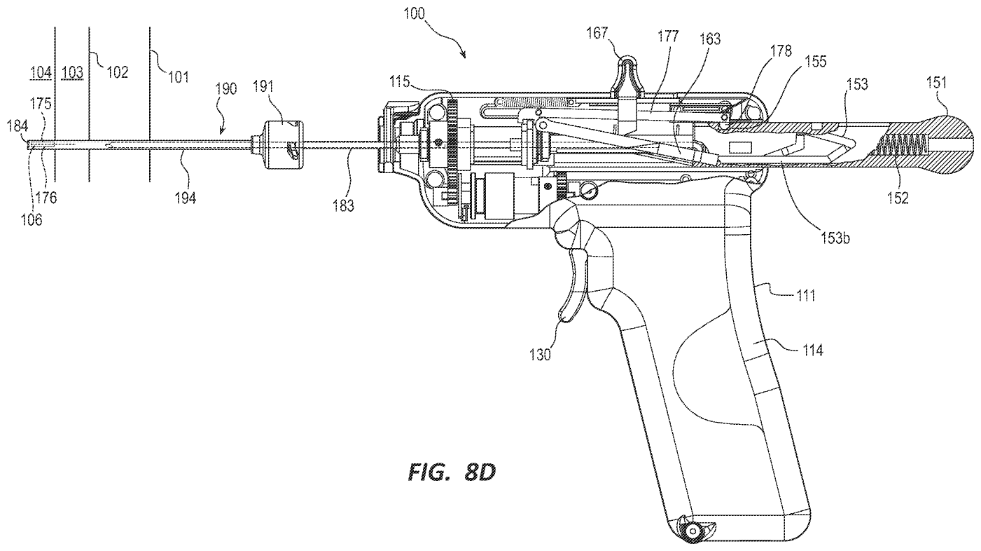

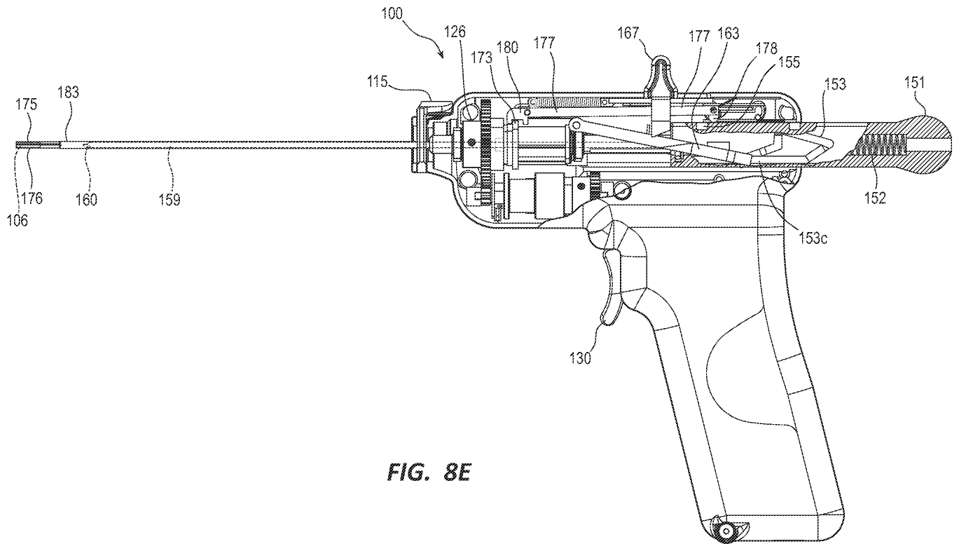

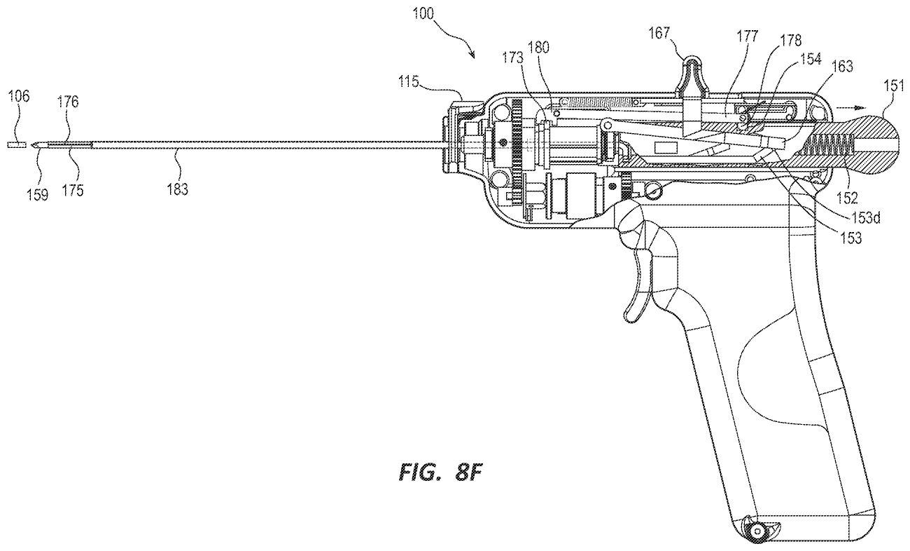

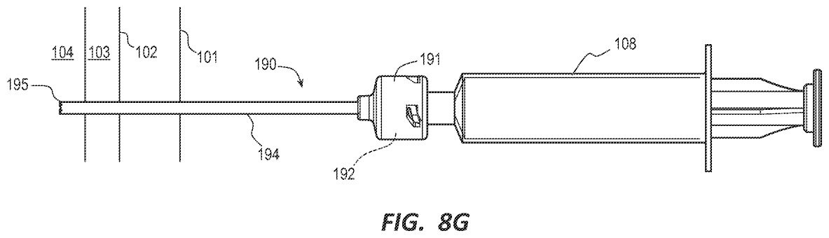

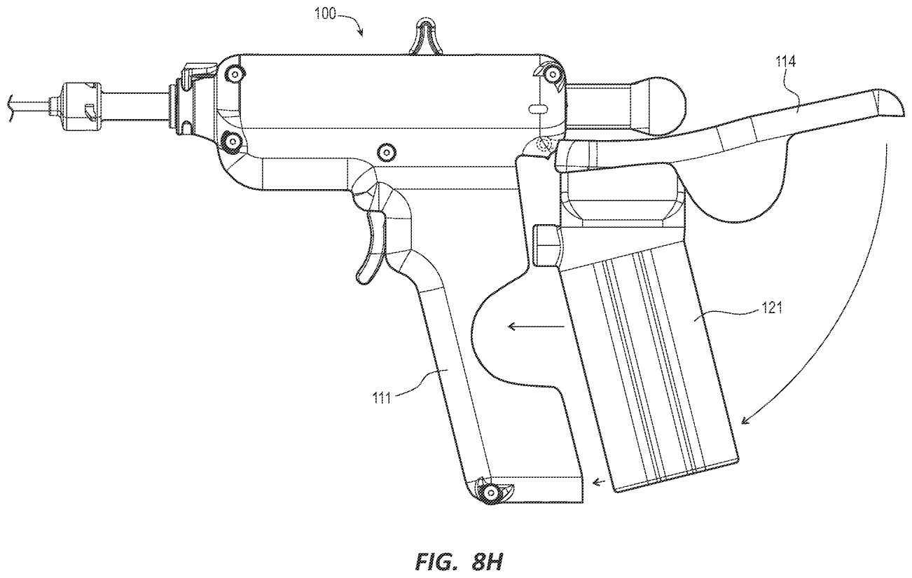

In the depicted embodiment, at least a portion of the powertrain assembly 120 can be disposed within the grip portion 113. The powertrain assembly 120 includes a reusable housing 121, a motor 122, a power source 123, and a controller 124, one or more of which may be disposed within the housing 121. The housing 121 and one or more components of the powertrain assembly 120 may be selectively removed from the handle housing 111 through a selectively openable door 114 following a bone biopsy procedure (as is shown in FIG. 8H). The housing 121 and one or more components of the powertrain assembly 120 can thereafter be charged (e.g., the power source 123 can be charged) and/or placed into a second bone biopsy device for use in a subsequent bone biopsy procedure.

The motor 122 may be any suitable type of rotatory motor. For example, the motor 122 may be a DC brushed motor, a DC brushless motor, a stepper motor, a servo motor, a pneumatic motor, or an AC powered motor, etc. The motor 122 may also be bi-directional. The motor 122 can include a drive shaft extending from the motor 122. The motor 122 may rotate the drive shaft at a speed ranging from about 0 rpm to about 50,000 rpm, or from about 15 rpm to about 20,000 rpm. The motor 122 can be electrically coupled to the power source 123 and to a motor activation switch 130 (e.g., trigger).

As depicted in the illustrated embodiment of FIG. 2, the power source 123 may include a single battery or a plurality of batteries. The battery or batteries may be replaceable or rechargeable. The battery or batteries can be recharged through a charging port at a base of the reusable housing 121. For example, the reusable housing 121 containing the battery or batteries can placed into a batter charging device between bone biopsy procedures. In some embodiments, the controller 124 may include a printed circuit board (PCB) that is electrically coupled to the power source 123, the motor 122, and the trigger 130. The controller 124 can be configured to control activation, rotation direction, and rotation speed of the motor 122 when the trigger 130 is actuated by the practitioner. In some embodiments, the PCB may be programmed to reverse the rotation direction of the motor 122 for a brief time when the trigger 130 is released by the practitioner.

As set forth above, in certain embodiments following a bone biopsy procedure, the motor 122, power source 123, and controller 124 (which can be contained within a reusable housing 121) may be selectively removed from the bone biopsy device 100 and the handle assembly 110. The bone biopsy device 100, handle assembly 110, and outer coax assembly 190 may thereafter be disposed of in a safe manner. When removed, the motor 122, power source 123, and/or controller 124 may be refurbished for use in a subsequent procedure. Refurbishment may include cleaning, sterilizing, recharging, or replacing the motor 122, power source 123, and/or controller 124.

Referring to FIGS. 2-5 and 7, the powertrain assembly 120 may include a clutch system 131 and a drive train 126. In the illustrated embodiment, the clutch system 131 includes a driver 133, a sleeve 136, an axle 140, and a resilient member 141 (e.g., compression spring). The driver 133 can be coupled to a proximal gear 132 that engages with and is rotationally driven by a pinion gear 125 coupled to and rotationally driven by the motor 122. The driver 133 includes one or more arcuate driver ramps 134. The sleeve 136 can be configured to slidingly receive the driver 133. One or more sleeve ramps 137 are disposed within the sleeve 136. The driver ramps 134 can engage with the sleeve ramps 137 to displace the sleeve 136 distally or away from the driver 133 when the clutch system 131 is rotated in a first direction. When the sleeve 136 is displaced the resilient member 141 is compressed and the sleeve 136 engages with a clutch gear 139 resulting in rotation of the clutch gear 139 by the motor 122 via the clutch system 131. When the clutch system 131 is rotated in a second direction, opposite of the first direction, the resilient member 141 can apply a proximally directed force to the sleeve 136 to proximally displace the sleeve 136 toward the driver 133 as the driver ramps 134 engage with the sleeve ramps 137. When the sleeve 136 is displaced proximally, the sleeve 136 disengages from the clutch gear 139 resulting in free rotation of the clutch gear 139. The proximal gear 132, driver 133, and clutch gear 139 may be fixedly coupled to the axle 140.

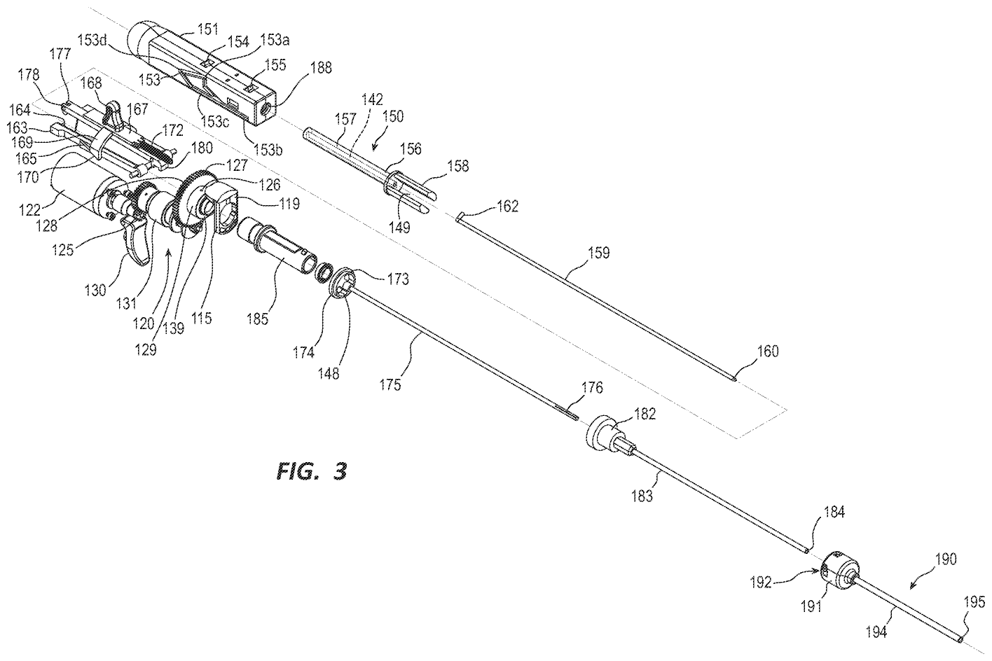

In the embodiment illustrated in FIGS. 2 and 3, the clutch gear 139 engages with and rotationally drives the drive train 126 operably coupled to the tissue sampling assembly 150. The drive train 126 includes a drive train gear 127, a proximal portion 128 extending proximally from the drive train gear 127 to couple with and rotate a trocar hub 156 and a distal portion 129 extending distally from the drive train gear 127 to couple with and rotate the spacer 185.

Referring to FIGS. 2 and 3, the tissue sampling assembly 150 includes a trocar displacement member or extension member 151, a trocar hub 156, a penetration member 159 (e.g., trocar), a track arm 163, a slider 167, an inner cannula 175, an inner cannula displacement member 177, and an intermediate cannula 183.

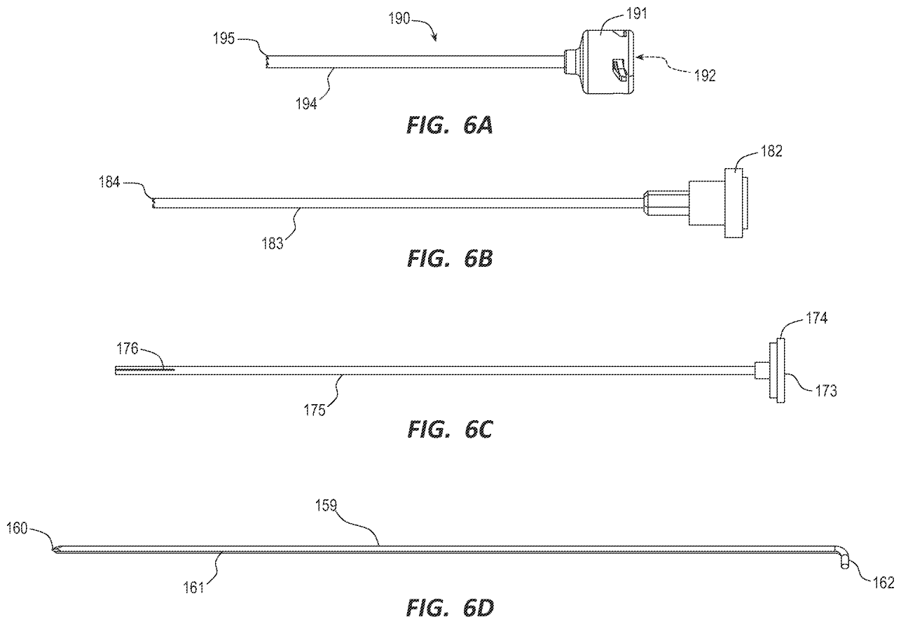

Referring to FIGS. 2, 3, and 6D, the trocar 159 is an elongate rod having a penetrating tip 160. The penetrating tip 160 may include a plurality of facets with cutting edges. The cutting edges may be angled to allow for drilling of the trocar 159 into a bone or other hard or rigid tissue. In some embodiments, the penetrating tip 160 may include spiral flutes. A laterally extending protrusion 162 is disposed adjacent a proximal end of the trocar 159. In the depicted embodiment, a proximal end of the trocar 159 is bent at an approximately 90-degree angle relative to a longitudinal axis of the trocar 159 to form the lateral protrusion 162. In some embodiments, the laterally extending protrusion 162 may be a pin oriented transverse to a longitudinal axis of the trocar 159. The protrusion 162 extends through a longitudinal slot 142 of a proximal portion 157 of the trocar hub 156 and is coupled to the trocar displacement member 151 such that the trocar 159 is rotatable relative to the trocar displacement member 151. A distal portion 158 of the trocar hub 156 extends proximally from and is engaged with the proximal portion 128 of the drive train 126 such that the trocar hub 156 and the trocar 159 are rotated by the drive train 126. In certain embodiments, the trocar 159 may include a longitudinally extending groove or trough 161 as shown in FIG. 6D. The groove 161 may have a substantially V-shape or U-shape and be configured for passage of a guidewire through a lumen of the inner cannula 175.

As illustrated in the embodiment of FIGS. 2 and 3, the trocar displacement member 151 is slidingly coupled to and extends proximally from the upper portion 112 of the handle housing 111. The trocar displacement member 151 is also slidingly coupled to the proximal portion 157 of the trocar hub 156. The lateral protrusion 162 of the trocar 159 is disposed in an annular groove 188 such that the trocar displacement member 151 can be longitudinally displaced by and be rotated relative to the trocar displacement member 151. A compression spring 152 may be disposed within the trocar displacement member 151 to apply a proximally directed force to a distal end wall of the trocar displacement member 151 to proximally displace the trocar displacement member 151 relative to the trocar hub 156. The trocar displacement member 151 may include a passage through the distal end wall in axial alignment with the groove 161 of the trocar 159 and configured for passage of a guidewire through the bone biopsy device 100 when in use.

A guide track 153 may be disposed on at least one lateral side of the trocar displacement member 151. The guide track 153 can include a plurality of segments, a first track segment 153a, a second track segment 153b, a third track segment 153c, and a fourth track segment 153d to guide movement of a track arm 163 when the trocar displacement member 151 is longitudinally displaced relative to the track arm 163. The track arm 163 may include forked arms configured to extend along lateral sides of the trocar displacement member 151. A protrusion 164 extends radially inward from each proximal end of the forked arms. The protrusions 164 engage with the guide track 153 and are guided through the track segments 153a, 153b, 153c, 153d to control longitudinal movement of trocar displacement member 151. For example, the protrusions 164 can be guided from 153a to 153b as the trocar displacement member 151 is displaced proximally and from 153b to 153c as the trocar displacement member 151 is displaced distally. A distal end of the track arm 163 is pivotably coupled to the handle housing 111.

A proximal recess 154 and a distal recess 155 are disposed in a top surface of the trocar displacement member 151 to selectively receive a protrusion 178 extending downward from a proximal end of the inner cannula displacement member 177. The inner cannula displacement member 177 can include an engagement portion 180 disposed at a distal end and configured to selectively engage with a flange 174 of the inner cannula hub 173 to longitudinally displace the inner cannula 175. The engagement portion 180 can include a recess disposed between two downwardly extending legs. A torsion spring 181 is coupled to the proximal end of the inner cannula advancement member 177 to bias the protrusion 178 into the recesses 154, 155.

A slider 167 may be slidingly coupled to the handle housing 111. A grip 168 configured to be gripped or otherwise engaged by a hand of a user can extend through a longitudinal slot of the handle housing 111. A saddle portion 169 can extend downwardly from the grip 168 and at least partially surround the trocar displacement member 151. A proximally facing ramp 170 may be disposed on each leg of the saddle portion 169. The ramps 170 may be configured to engage with distally facing ramps 165 of the track arm 163 when the slider 167 is moved from a distal position to a proximal position. When the ramps 170 engage with the ramps 165, the proximal end of the track arm 163 is displaced downwardly within the track 153. A tension spring 172 may be coupled to the slider 167 and to the handle housing 111 to bias the slider 167 distally.

As illustrated in FIGS. 3 and 6C, the inner cannula 175 includes a tubular shaft having a lumen extending therethrough allowing the trocar 159 to be coaxially disposed within the inner cannula 175. A distal portion of the inner cannula 175 includes at least one slot 176 through a wall of the shaft. A plurality of slots 176 can also be used (e.g., three slots 176 disposed around the shaft). The slot 176 allows the distal portion to radially expand when a core tissue sample is ejected from the inner cannula 175, allowing the core tissue sample to be ejected with minimized damage. A proximal end of the shaft is fixedly coupled to the inner cannula hub 173. The inner cannula hub 173 is slidingly coupled to the distal portion 158 of the trocar hub 156 to allow the inner cannula hub 173 to be moved from a proximal position to a distal position by the inner cannula advancement member 177. A tab 148 of the inner cannula hub 173 is disposed within a slot 149 of the distal portion 158 of the trocar hub 156 to cause rotation of the inner cannula hub 173 and the inner cannula 175 when the trocar hub 156 is rotated by the drive train 126.

As illustrated in FIG. 6B, the intermediate cannula 183 includes a tubular shaft having a lumen extending therethrough allowing the inner cannula 175 to be coaxially disposed within the intermediate cannula 183. A distal end of the intermediate cannula 183 includes a hole cutting tip 184. In certain embodiments the tip 184 can be in the form of a trephine tip having a plurality of teeth. A proximal end of the intermediate cannula 183 is fixedly coupled to an intermediate cannula hub 182. The intermediate cannula hub 182 is a cylinder and fixedly coupled to the drive train 126 such that the intermediate cannula 183 is rotated by the drive train 126.

As illustrated in FIGS. 2 and 3, the spacer 185 may be selectively coupled to the handle housing 111. The spacer 185 includes a lumen extending therethrough and configured to allow passage of the trocar 159, inner cannula 175, and intermediate cannula 183. A proximal portion of the spacer 185 engages with the distal portion 129 of the drive train 126 such that the spacer 185 can be rotated by the drive train 126. The distal portion 129 of the drive train 126 includes a male hex shape and the proximal portion of the spacer 185 includes a female hex shape configured to receive the hex shaped distal portion 129. A clip 115 selectively couples the spacer 185 to the handle housing 111. The clip 115 includes a keyhole lock having an upper portion having diameter larger than a diameter of a proximal portion of the spacer 185 and a lower portion having a diameter smaller than the diameter of the proximal portion but larger than a recessed portion of the spacer 185. When the spacer 185 is coupled to the handle housing 111, the lower portion engages the spacer 185 to lock the spacer 185 into engagement with the handle housing 111. When the user desires to remove the spacer 185 from the handle housing 111, a finger tab 119 can be depressed causing the clip 115 to move downward and the upper portion 117 to move around the spacer 185 allowing the spacer 185 to be removed from the handle housing 111.

When the spacer 185 is coupled to the handle housing 111, the bone biopsy device 100 can be inserted into a patient to a first depth. When the spacer 185 is removed from the handle housing 111, the bone biopsy device 100 can be inserted into the patient to a second depth. A distance of the difference between the first insertion depth and the second insertion depth can be up to a length of the spacer 185. In some embodiments, the length of the spacer 185 may be shortened without removal from the handle housing 111, allowing for the second insertion depth to be deeper than the first insertion depth. For example, the spacer 185 may include a distal portion and a proximal portion that are threadingly coupled allowing for length adjustment by rotating the proximal portion relative to the distal portion.

As illustrated in FIG. 6A, the coax assembly 190 may be selectively coupled to a distal end of the spacer 185 via a coax connector 191 when the bone biopsy device 100 is in a ready state. The coax assembly 190 includes the coax connector 191 and an outer coax cannula 194. The coax connector 191 may include a female Luer fitting 192 for coupling to a medical device (e.g., syringe) to withdraw a tissue sample or infuse a fluid or medicament into the patient through the coax assembly 190. The coax connector 191 is coupled to the distal end of the spacer 185 in a way that allows the coax assembly 190 to be rotated by the spacer 185. In the illustrated embodiment, the coax connector 191 is coupled to the distal end of the spacer 185 using a bayonet-type connection where a partial rotation of the coax connector 191 is needed to disconnect from the spacer 185. In other embodiments, the coax connector 191 is coupled to the distal end of the spacer 185 using a clip have a similar configuration of the clip 115.

A proximal end of the outer coax cannula 194 is fixedly coupled to the coax connector 191. The outer coax cannula 194 includes a lumen extending therethrough allowing the intermediate cannula 182 to be coaxially disposed within the outer coax cannula 194. A distal end of the outer coax cannula 194 includes a hole cutting tip 195 configured to cut a hole in bone when the outer coax cannula 194 is rotated. In certain embodiments, the hole cutting tip 195 is a trephine tip having a plurality of serrated or jagged teeth.

In use, the bone biopsy device 100 can be used to obtain a core tissue sample from a bone lesion and/or bone marrow. FIGS. 8A-8H illustrate methods of use of the bone biopsy device 100 to obtain a core tissue sample from a bone lesion and/or bone marrow. FIG. 8A illustrates the bone biopsy device 100 in the ready state. The reusable housing 121 is disposed within the handle housing 111. The door 114 is closed to retain the reusable housing 121 within the handle housing 111 and to prevent contamination of the reusable housing 121 with body fluids. The spacer 185 is coupled to the handle housing 111 and the coax connector 191 of the coax assembly 190 is coupled to the spacer 185. The penetrating tip 160 of the trocar 159 extends distally beyond the outer coax cannula 194. Distal ends of the inner cannula 175 and the intermediate cannula 183 are positioned proximal to the trephine tip 195 of the outer coax cannula 194. The trocar displacement member 151 is in an intermediate position where the track arm 163 is disposed at the first track segment 153a of the track 153. The engagement portion 180 of the inner cannula displacement member 177 is in engagement with the inner cannula hub 173. The slider 167 is in a distal position. The clutch system 131 is disengaged from the drive train 126. In some embodiments, the bone biopsy device 100 may be disposed over a guidewire 109 that has been inserted through the skin 101 of a patient such that a distal end of the guidewire 109 is adjacent the bone periosteum 102. The guidewire 109 can extend through the inner cannula 175 via the trocar groove 161 (not shown) and through the trocar displacement member 151.

As depicted in FIG. 8B, the bone biopsy device 100 is activated to rotate the trocar 159 and the coax assembly 190 as the penetrating tip 160 and the trephine tip 195 are inserted through the skin 101, the bone periosteum 102, the bone cortex 103, and into the bone lesion and/or bone marrow 104. When rotated, the penetrating tip 160 and the trephine tip 195 can drill a hole through the bone periosteum 102 and the bone cortex 103. The trocar 159 may be optionally inserted into the patient over the guidewire 109 that passes through the inner cannula 175 via the trocar groove 161 as previously described. The guidewire 109 may have been inserted using any suitable known technique prior to insertion of the bone biopsy device 100. The guidewire 109 can then be removed prior to rotating the outer coax cannula 194 when the penetrating tip 160 is adjacent the bone periosteum 102. In other instances, rotation of the outer coax cannula 194 and trocar 159 can begin prior to removal of the guidewire 109 to facilitate insertion of the penetrating tip 160 and the trephine tip 195 through the skin 101.

When the bone biopsy device 100 is activated, the trigger 130 is displaced proximally by a user's finger causing electricity to flow from the power source 123 to the motor 122. When energized, the motor 122 rotates in the first direction causing the driver 133 of the clutch system 131 to rotate in the first direction. In some embodiments, the user can control the motor speed through the trigger 130. For example, the user may partially actuate the trigger 130 to run the motor 122 at a first speed and actuate the trigger 130 further to run the motor 122 at a second speed, third speed, fourth speed, etc. When the driver 133 is rotated, the driver ramps 134 (not shown) engage with the sleeve ramps 137 (not shown) causing the sleeve 136 to be displaced distally. When the sleeve 136 is displaced distally, the sleeve 136 engages with the clutch gear 139 to rotate the drive train 126 in the first direction. When the drive train 126 is rotated in the first direction, the trocar 159, the inner cannula 175, the intermediate cannula 183, and the outer coax cannula 194 are rotated in the first direction.

When the trephine tip 195 is in the bone lesion and/or bone marrow 104, the bone biopsy device 100 is de-activated by release of the trigger 130 by the finger of the user. When de-activated, the controller 124 (not shown) causes the motor 122 to briefly rotate in the second direction. When the motor rotates in the second direction, the driver 133 is rotated in the second direction. The spring 141 (not shown) applies a proximally directed force to the sleeve 136, causing the sleeve 136 to move proximally and disengage the clutch gear 139. When the clutch gear 139 is disengaged, the drive train 126 can be freely rotated, not allowing the trocar 159, the inner cannula 175, the intermediate cannula 183, and the outer coax cannula 194 to be rotated via the handle assembly 110.

FIG. 8C illustrates the bone biopsy device 100 in a pre-biopsy state where the bone biopsy device 100 is decoupled from the coax connector 191 and removed from the coax assembly 190 while the outer coax cannula 194 remains inserted in the patient. The spacer 185 (not shown) is decoupled from the handle housing 111 by depression of the clip 115 and removal from the bone biopsy device 100. In other embodiments, the spacer 185 (not shown) may be left coupled to the device 100 or otherwise not be removed from the bone biopsy device 100 and a sample may be obtained. The trocar 159 is retracted or displaced proximally within the inner cannula 175 when the slider 167 is moved proximally. When the slider 167 is moved proximally, the slider ramp 170 engages the track arm ramp 165, causing the track arm 163 to be displaced downwardly within the track 153. When displaced downwardly, the track arm 163 is guided to the second track segment 153b when the spring 152 applies a proximally directed force to the trocar displacement member 151 causing the trocar displacement member 151 to move proximally relative to the handle housing 111. When the trocar displacement member 151 moves proximally, the trocar 159 is moved proximally, resulting in the penetrating tip 160 being positioned proximally to the trephine tip 184 of the intermediate cannula 183. Additionally, when the trocar displacement member 151 moves proximally, the proximal protrusion 178 of the inner cannula displacement member 177 engages with the distal recess 155 of the trocar displacement member 151. The spring 172 coupled to the slider 167 causes the slider 167 to return to its ready state when the track arm 163 is positioned in the second track segment 153b.

FIG. 8D illustrates the bone biopsy device 100 in a biopsy state where the bone biopsy device 100 is re-inserted into the patient through the coax assembly 190 such that the intermediate cannula 183 and the inner cannula 175 extend beyond the outer coax cannula 194 and into the bone lesion and/or bone marrow 104. As the inner cannula 175 and intermediate cannula 183 are inserted into the bone lesion and/or bone marrow 104, the bone biopsy device 100 is activated, as previously described, causing the inner cannula 175 and the intermediate cannula 183 to rotate in the first direction. As the cannulae 175, 183 are inserted and rotated, the trephine tip 184 of the intermediate cannula 183 cuts a hole in the bone lesion and/or bone marrow 104 causing a core tissue sample 106 to be collected within the inner cannula 175.

FIG. 8E illustrates the bone biopsy device 100 in a sample ejection ready state where the bone biopsy device 100 is removed from the patient and from the coax assembly 190. The inner cannula 175 extends beyond the intermediate cannula 183. The trocar displacement member 151 is moved distally by a user's hand. When the trocar displacement member 151 is moved distally by a user's hand, the track arm 163 is guided to the third track segment 153c. The inner cannula displacement member 177 is moved distally by the trocar displacement member 151, causing the inner cannula hub 173 to move distally when the engagement portion 180 engages with the inner cannula hub 173. When the inner cannula hub 173 moves distally, the inner cannula 175 moves distally such that the slot 176 extends beyond the intermediate cannula 183. The trocar 159 moves distally such that the penetrating tip 160 remains within the inner cannula 175. When the inner cannula hub 173 is fully distally displaced such that it contacts the drive train 126, a radius of the protrusion 178 of the inner cannula displacement member 177 allows the protrusion 178 to be displaced from the distal recess 155. This allows the trocar displacement member 151 to be further distally displaced.

FIG. 8F illustrates the bone biopsy device 100 in a sample ejection state where the trocar 159 is moved distally to push or eject the core tissue sample 106 from the inner cannula 175. The trocar displacement member 151 is further moved distally by the user's hand causing the track arm 163 to move to a fourth track segment 153d. The trocar 159 is moved distally to a fully extended position causing the penetrating tip 160 to engage with and eject the core tissue sample 106 from the inner cannula 175. As the core tissue sample 106 is ejected, the slot 176 may allow the inner cannula 175 to radially expand, resulting in less required force applied to the trocar displacement member 151 by the user's hand to eject the core tissue sample 106 when compared to core tissue sample ejection without a slot 176.

Following core tissue sample ejection, the trocar displacement member 151 is displaced proximally by the spring 152 as the track arm 163 moves from the fourth track segment 153d to the first track segment 153a. The inner cannula displacement member 177 engages the proximal recess 154 to move the inner cannula 175 proximally from the core tissue sample ejection position to a retracted position. In this configuration, the bone biopsy device 100 is returned to its ready state

In some instances, as depicted in FIG. 8G, an aspiration device (e.g., syringe, vacuum sample collection tube, or pump, etc.) 108 may be used to obtain a tissue sample of the bone lesion and/or bone marrow 104. For example, the aspiration device 108 can be coupled to the Luer fitting 192 of the coax connector 191. The aspiration device 108 can then be used to aspirate a tissue sample of the bone lesion and/or bone marrow 104 through the needle.

In certain embodiments, as illustrated in FIG. 8H, following the bone biopsy procedure the selectively openable door 114 may be opened and the reusable housing 121 removed from the handle housing 111 for refurbishment of one or more components thereof as previously described.

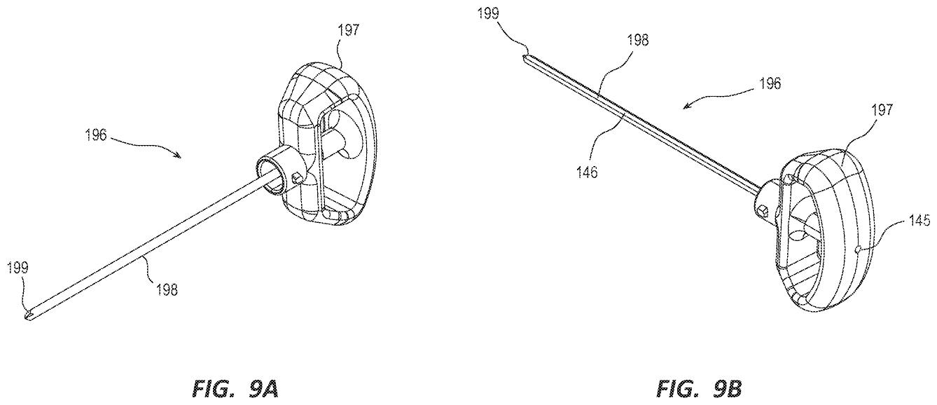

In certain instances, a trocar assembly 196 may be selectively coupled to the coax assembly 190 to facilitate manual positioning of the coax assembly 190 prior to using the bone biopsy device 100. As illustrated in FIGS. 9A and 9B, the trocar assembly 196 can include a handle member 197 and a trocar 198. In use, the trocar 198 may be inserted into the coax connector 191 and through the outer coax cannula 194 such that a penetrating tip 199 of the trocar 198 extends beyond the outer coax cannula 194. The handle member 197 may also be coupled to the coax connector 191. The trocar assembly 196 and coax assembly 190 can then be moved and/or placed into a desired location (e.g., moved through the soft tissue). After proper placement is achieved, the trocar assembly 196 can be removed by uncoupling the handle member 197 from the coax connector 191 and removing the trocar 198 from the outer coax cannula 194. The powered bone biopsy device 100 can thereafter be coupled with the outer coax cannula 194 and used to obtain a biopsy sample as previously described. In certain embodiments, the handle member 197 may include a guidewire passage 145 in communication with a groove 146 of the trocar 198. The guidewire assembly 196 may be inserted into the patient over a previously inserted guidewire with the guidewire passing through the groove 146 and the guidewire passage 145.

In other embodiments, the trocar assembly 196 can be used to reposition or redirect the coax assembly 190 within the bone lesion and/or bone marrow to obtain subsequent tissue samples. For instance, after using the bone biopsy device 100 (as previously discussed), the trocar assembly 196 can be inserted into and coupled to the coax assembly 190 to aid in manually repositioning and/or redirecting the coax assembly 190 prior to obtaining a subsequent core tissue sample or tissue sample using the bone biopsy device 100 or an aspiration device 108.

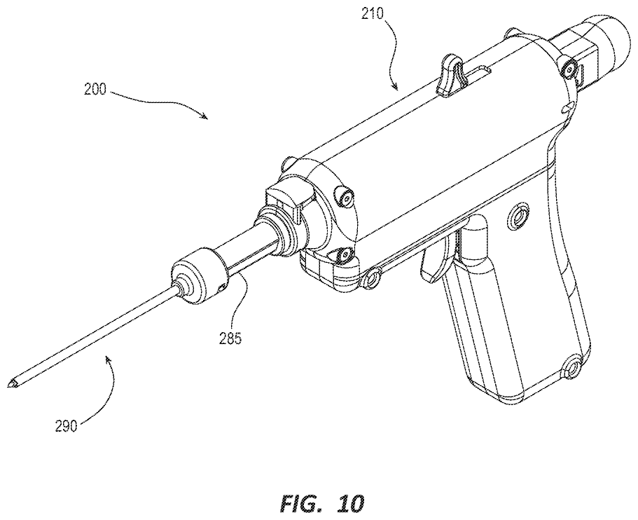

FIGS. 10-12 depict an embodiment of a bone biopsy device 200 that resembles the bone biopsy device 100 described above in certain respects. Accordingly, like features are designated with like reference numerals, with the leading digit incremented to “2.” For example, the embodiment depicted in FIGS. 10-12 includes a handle assembly 210 that may, in some respects, resemble the handle assembly 110 of FIG. 1. Relevant disclosure set forth above regarding similarly identified features thus may not be repeated hereafter. Moreover, specific features of the bone biopsy device 100 and related components shown in FIGS. 1-8H may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the bone biopsy device 200 and related components depicted in FIGS. 10-12. Any suitable combination of the features, and variations of the same, described with respect to the bone biopsy device 100 and related components illustrated in FIGS. 1-8H can be employed with the bone biopsy device 200 and related components of FIGS. 10-12, and vice versa. This pattern of disclosure applies equally to further embodiments depicted in subsequent figures and described hereafter, wherein the leading digits may be further incremented

FIGS. 10-12 illustrate another bone biopsy device 200. The illustrated embodiment of the bone biopsy device 200 of FIG. 10 includes a handle assembly 210, a coax assembly 290, and a spacer 285 selectively disposed between the handle assembly 110 and the coax assembly 190.

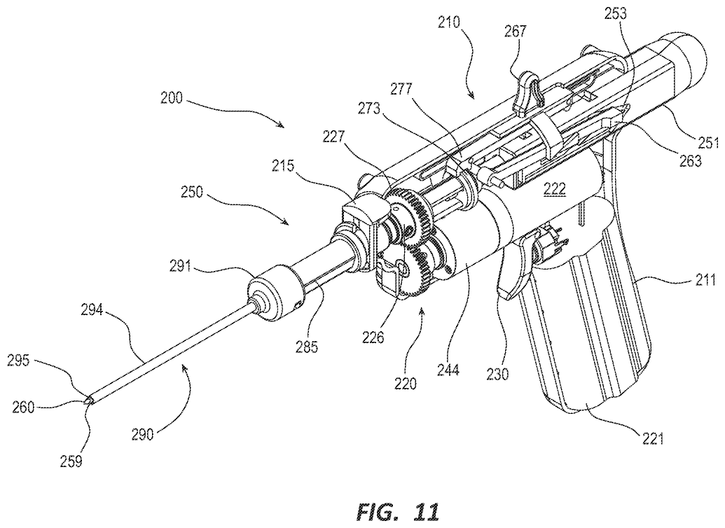

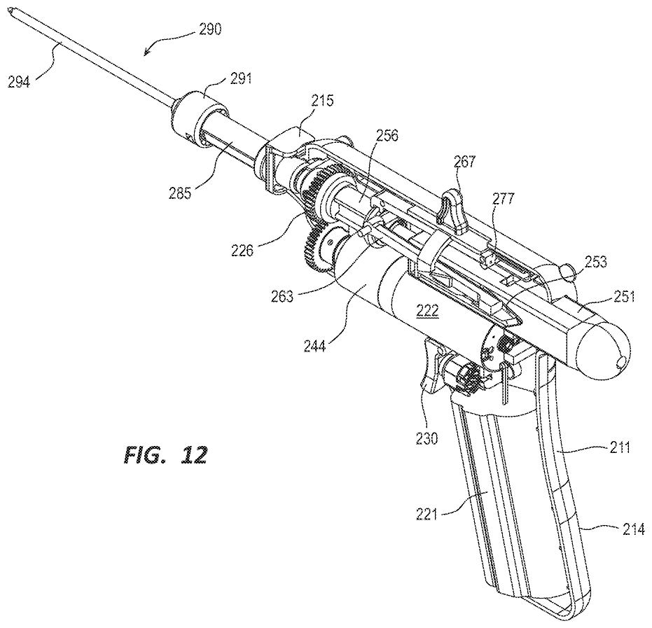

FIGS. 11 and 12 illustrate the handle assembly 210 includes a powertrain assembly 220, a tissue sampling assembly 250, a spacer 285, and a coax assembly 290. The components and functions of the tissue sampling assembly 250, the spacer 285, and the coax assembly 290 are substantially similar to the components and functions of the tissue sampling assembly 150, the spacer 185, and the coax assembly 190, respectively. With regards to the powertrain assembly 220, FIGS. 11 and 12 depict the powertrain assembly 220 includes a motor 222, a power source, and a controller disposed within a reusable housing 221.

Referring to FIGS. 11 and 12, the powertrain assembly 220 may include a gear box 244 operably coupled to the motor 222 and to the drive train 226. The gear box 244 may include a plurality of gears configured to increase or decrease rotational speeds of the drive train 226, the trocar 259, an inner cannula, an intermediate cannula, the spacer 285, and the trocar assembly 296 relative to the rotational speed of the motor 222. For example, in one embodiment, the plurality of gears within the gear box 244 may be sized and arranged such that the rotational speeds of the drive train 226, the trocar 259, the inner cannula, the intermediate cannula, the spacer 285, and the coax assembly 290 are slower than the rotational speed of the motor 222. In another embodiment, the plurality of gears within the gear box 244 may be sized and arranged such that the rotational speeds of the drive train 226, the trocar 259, the inner cannula, the intermediate cannula, the spacer 285, and the coax assembly 290 are faster than the rotational speed of the motor 222.

Similarly, as shown for the bone biopsy device 100 in FIG. 8H, a selectively openable door 214 may be opened following a bone biopsy procedure. A reusable housing 221 containing one or more of a power source, a controller, the motor 222, and the gear box 244 may thereafter be removed. In some instances, one or more of the power source, controller, motor 222, and gear box 244 are not contained within the reusable housing 221. Such components can also be removed as desired. When removed, the reusable housing 221 (and/or one or more components) may be refurbished for use in a subsequent procedure. Refurbishment may include cleaning, sterilizing, recharging, or replacing the motor 222, gear box 244, power source, and/or controller.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of obtaining a core tissue sample from a patient may include one or more of the following steps: setting a bone biopsy device to a ready state; activating the bone biopsy device, wherein an outer coax cannula, an inner cannula, an intermediate cannula, and a penetration member are rotated during insertion to a first position in the patient; removing the inner cannula, the intermediate cannula, and the trocar from the outer coax cannula, wherein the outer coax cannula remains inserted in the patient; removing a spacer from the bone biopsy device; retracting the trocar from a first extended position to a retracted position; reinserting the inner cannula, the intermediate cannula, and the trocar into the outer coax cannula; activating the bone biopsy device, wherein the inner cannula, the intermediate cannula, and the trocar are rotated; further inserting the inner cannula and the intermediate cannula to a second position, wherein a first core tissue sample is obtained within the inner cannula; removing the inner cannula, the intermediate cannula, and the trocar from the patient; displacing the inner cannula to extend from the intermediate cannula; and displacing the trocar from the retracted position to a second extended position to eject the first core tissue sample from the inner cannula. Other steps are also contemplated.

The phrase “coupled to” refers to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The directional terms “distal” and “proximal” are given their ordinary meaning in the art. That is, the distal end of a medical device means the end of the device furthest from the user during use. The proximal end refers to the opposite end, or the end nearest the user during use. As specifically applied to the bone biopsy device, the proximal end of the device refers to the end nearest the handle housing and the distal end refers to the opposite end, the end nearest the end of the outer coax cannula. Thus, if at one or more points in a procedure the user changes the orientation of the device, as used herein, the term “proximal end” always refers to the handle housing end of the device (even if the distal end is temporarily closer to the user).

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers.

In the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

The terms “a” and “an” can be described as one, but not limited to one.

Unless otherwise stated, all ranges include both endpoints and all numbers between the endpoints.

Recitation in the claims of the term “first” with respect to a feature or element does not necessarily imply the existence of a second or additional such feature or element. Elements recited in means-plus-function format are intended to be construed in accordance with 35 U.S.C. § 112 6. It will be apparent to those having skill in the art that changes may be made to the details of the above-described embodiments without departing from the underlying principles of the invention. Embodiments of the invention in which an exclusive property or privilege is claimed are defined as follows.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A bone biopsy device, comprising:
a handle housing;
a powertrain assembly;
a tissue sampling assembly configured to be rotated about a longitudinal axis by the powertrain assembly;
a coax assembly selectively couplable to the handle housing; and
a spacer longitudinally disposed between the handle housing and the coax assembly and selectively coupleable to the handle housing and the coax assembly.

2. The bone biopsy device of claim 1, wherein the tissue sampling assembly comprises:
an intermediate cannula configured to cut a hole in a tissue;
an inner cannula coaxially disposed within the intermediate cannula and configured to receive a tissue sample;
a trocar coaxially disposed within the inner cannula; and
a trocar displacement member coupled to the trocar and configured to longitudinally displace the trocar relative to the inner cannula.

3. The bone biopsy device of claim 2,
wherein the inner cannula comprises at least one slot extending through a wall of a distal portion, and
wherein the at least one slot is configured to allow radial expansion of the distal portion.

4. The bone biopsy device of claim 2,
wherein the tissue sampling assembly further comprises an inner cannula displacement member operably coupled to the trocar displacement member, and
wherein the inner cannula displacement member is configured to longitudinally displace the inner cannula relative to the intermediate cannula.

5. The bone biopsy device of claim 2, wherein the trocar comprises:
a longitudinal groove configured to receive a guidewire; and
a multi-faceted penetrating tip.

6. The bone biopsy device of claim 2, wherein the trocar displacement member comprises:
a guide track comprising a plurality of segments; and
a proximal recess and a distal recess for engagement with an inner cannula displacement member.

7. The bone biopsy device of claim 6, further comprising a track arm coupleable to the guide track and configured to control longitudinal movement of the trocar.

8. The bone biopsy device of claim 7,
wherein the plurality of segments of the guide track comprise:
a first segment;
a second segment;
a third segment; and
a fourth segment;
wherein when the track arm is disposed in the first segment, the trocar is in a first extended position;
wherein when the track arm is disposed in the second segment, the trocar is in a first retracted position;
wherein when the track arm is disposed in the third segment, the trocar is in a second retracted position; and
wherein when the track arm is disposed in the fourth segment, the trocar is in a second extended position.

9. The bone biopsy device of claim 2, further comprising an inner cannula displacement member,
wherein the trocar displacement member comprises a proximal recess and a distal recess configured to engage with the inner cannula displacement member,
wherein the inner cannula displacement member distally displaces the inner cannula to an extended position when the inner cannula displacement member is engaged with the distal recess, and
wherein the inner cannula displacement member proximally displaces the inner cannula to a retracted position when the inner cannula displacement member is engaged with the proximal recess.

10. The bone biopsy device of claim 8, further comprising a slider comprising a proximally facing ramp,
wherein the track arm comprises a distally facing ramp, and
wherein the track arm is displaced from the first track segment to the second track segment when the slider is displaced proximally causing the proximally facing ramp to slidingly engage with the distally facing ramp.

11. The bone biopsy device of claim 1, wherein the powertrain assembly comprises a reusable component comprising:
a motor;
a power source; and
a controller,
wherein the motor, power source, and controller are disposed within a housing, and
wherein the housing is removable from the handle housing.

12. The bone biopsy device of claim 1,
wherein the powertrain assembly comprises a clutch configured to selectively rotate at least a portion of the tissue sample assembly and the coax assembly.

13. The bone biopsy device of claim 12, wherein the clutch comprises:
a sleeve comprising sleeve ramps;
a driver comprising driver ramps,
wherein the sleeve ramps operably engage with the driver ramps to displace the sleeve into engagement with a clutch gear when the sleeve and driver are rotated in a first direction,
and wherein the sleeve ramps operably engage with the driver ramps to displace the sleeve away from engagement with the gear when the sleeve and the driver are rotated in a second direction.

14. The bone biopsy device of claim 1, wherein the coax assembly comprises:
a connector; and
a coax cannula coupled to the connector and comprising a trephine tip.

15. A bone biopsy system, comprising:
a bone biopsy device comprising:
a handle housing;
a tissue sampling assembly configured to be rotated about a longitudinal axis;
a coax assembly selectively couplable to the handle housing;
a spacer longitudinally disposed between the handle housing and the coax assembly and selectively coupled to the handle and the coax assembly; and
a powertrain assembly comprising a reusable portion and a disposable portion.

16. The bone biopsy system of claim 15, wherein the reusable portion of the powertrain assembly comprises:
a housing containing:
a motor configured to rotate at least a portion of the tissue sampling assembly about a longitudinal axis;
a power source configured to power the motor; and
a controller configured to control rotation speed and rotation direction of the motor.

17. The bone biopsy system of claim 15, wherein the disposable portion of the powertrain assembly comprises a clutch configured to selectively engage with a drive train.

18. The bone biopsy system of claim 15, further comprising a trocar assembly comprising:
a handle configured to be gripped by a user; and
a trocar coupled to the handle.

19. The bone biopsy system of claim 15, further comprising an aspiration device comprising a male Luer fitting coupleable to a female Luer fitting of a connector of the coax assembly.

20. A method of obtaining a core tissue sample from a patient, comprising:
setting a bone biopsy device to a ready state;
activating the bone biopsy device, wherein an outer coax cannula, an inner cannula, an intermediate cannula, and a trocar rotate during insertion to a first position in the patient;
removing the inner cannula, the intermediate cannula, and the trocar from the outer coax cannula, wherein the outer coax cannula remains inserted in the patient;
removing a spacer from the bone biopsy device;
retracting the trocar from a first extended configuration to a retracted configuration;
reinserting the inner cannula, the intermediate cannula, and the trocar into the outer coax cannula;
activating the bone biopsy device, wherein the inner cannula, the intermediate cannula, and the trocar rotate;
further inserting the inner cannula and the intermediate cannula to a second position, wherein a first core tissue sample is obtained within the inner cannula;
removing the inner cannula, the intermediate cannula, and the trocar from the patient;
displacing the inner cannula relative to the intermediate cannula to extend from the intermediate cannula; and
displacing the trocar relative to the inner cannula from the retracted configuration to a second extended configuration to eject the first core tissue sample from the inner cannula.
